(19) United States
(12) Patent Application Publication
(10) Pub. No.: US 2005/0159485 A1
(43) Pub. Date: Jul. 21, 2005

(54) Combinations for the treatment of immunoinflammatory disorders and proliferative skin diseases

(76) Inventors: Edward Roydon Jost-Price, West Roxbury, MA (US); Palaniyandi Manivasakam, West Roxbury, MA (US); Grant R. Zimmermann, Somerville, MA (US); Nicole Hurst, Boston, MA (US); Jason Fong, Philadelphia, PA (US); Curtis Keith, Boston, MA (US); Alexis Borisy, Arlington, MA (US)

Correspondence Address: CLARK & ELBING LLP, 101 FEDERAL STREET, BOSTON, MA, 02110, US

(21) Appl. No.: 10/500,544

(22) PCT Filed: Jan. 2, 2003
(86) PCT No.: PCT/US03/00118

(30) Foreign Application Priority Data
Jan. 4, 2002 (US) 60345285

(51) Int. Cl. A61K 31/47; A61K 31/203; A61K 31/557

(52) U.S. Cl. 514/559; 514/573; 514/725; 514/311

(57) ABSTRACT

The invention features a method for treating a patient who has an immunoinflammatory disorder or a proliferative skin disease, or is at risk for developing an immunoinflammatory disorder or a proliferative skin disease, by administering to the patient a prostaglandin and a retinoid simultaneously or within 14 days of each other in amounts sufficient to reduce or inhibit immunoinflammatory or dermal/epidermal proliferation.

### BACKGROUND OF THE INVENTION

The invention relates to the treatment of immunoinflammatory disorders and proliferative skin diseases.

Immunoinflammatory disorders (e.g., rheumatoid arthritis, psoriasis, ulcerative colitis, Crohn's disease, stroke-induced brain cell death, septic shock syndrome, ankylosing spondylitis, fibromyalgia, inflammatory dermatoses, asthma, multiple sclerosis, type I diabetes, systemic lupus erythematosus, scleroderma, systemic sclerosis, and Sjögren's syndrome) are characterized by dysregulation of the immune system and inappropriate activation of body's defenses, resulting in damage to healthy tissue.

One percent of humans world-wide are afflicted with rheumatoid arthritis, a relentless, progressive disease causing severe swelling, pain, and eventual deformity and destruction of joints. According to the Arthritis Foundation, rheumatoid arthritis currently affects over two million Americans, of which women are three times more likely to be afflicted. Rheumatoid arthritis is characterized by inflammation of the lining of the joints and/or other internal organs, and the presence of elevated numbers of lymphocytes and high levels of proinflammatory cytokines.

Treatment of rheumatoid arthritis generally includes administration of (i) non-steroidal anti-inflammatories (e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate); (ii) steroids, (e.g., cortisone, dexamethasone, hydrocortisone, methylprednisolone, prednisolone, prednisone, triamcinolone); (iii) DMARDs, i.e., disease modifying antirheumatic drugs, (e.g., cyclosporine, azathioprine, methotrexate, leflunomide, cyclophosphamide, hydroxychoroquine, sulfasalazine, D-penicillamine, minocycline, and gold); or (iv) recombinant proteins (e.g., entanercept; soluble TNF receptor) and remicade (infliximab)).

Psoriasis is a common chronic proliferative skin disease, affecting up to 2% of the population. One characteristic of psoriasis is a strong hyperproliferation of epidermal keratinocytes and an incomplete epidermal differentiation that leads to severe scaling of the affected skin areas. This proliferative event is accompanied by an inflammation of the epidermis and dermis, with infiltrates of T-cells, neutrophils, and macrophages. Consequently, psoriasis has characteristics of both an autoimmune disease and a proliferative skin disease.

### SUMMARY OF THE INVENTION

We have discovered that the combination of a prostaglandin, alprostadil (also known as prostaglandin E1; (11α, 13E, 15S)-11,15-dihydroxy-9-oxoprost-13-enoic acid; 11α, 15α-dihydroxy-9-oxo-13-trans-prostenoic acid; or 3-hydroxy-2-(3-hydroxy-1-octenyl)-5-oxocyclopentaneheptanoic acid), and a retinoid, tretinoin (also known as vitamin A; all trans retinoic acid; or 3,7-dimethyl-9-(2,6,6-trimethylcyclohex-1-enyl)nona-2,4,6,8-all-trans-tetraenoic acid), brings about substantial suppression of TNFα levels induced in white blood cells. TNFα is a major mediator of inflammation. Specific blockade of TNF using antibodies or soluble receptors is a potent treatment for patients having an immunoinflammatory disease, such as inflammatory bowel disease or rheumatoid arthritis, or a proliferative skin disease. Thus, this combination can be used to treat immunoinflammatory disorders and proliferative skin diseases. Moreover, based on the shared action among prostaglandin family members and among retinoid family members, alprostidil and/or tretinoin can be replaced by a family member in the combination.

Accordingly, the invention features a method for treating a patient who has, or is at risk for developing, an immunoinflammatory disorder or proliferative skin disease, by administering to the patient (i) a prostaglandin; and (ii) a retinoid, in amounts that treat the patient. The prostaglandin and the retinoid may be administered separately or as components of a pharmaceutical composition.

The prostaglandin and retinoid can be administered within ten days of each other (e.g, within five days, twenty-four hours, or one hour of each other, or even simultaneously). Administration of each compound can occur 1-4 times each day, or as necessary to alleviate symptoms.

The specific amounts of prostaglandin and retinoid administered depend on the specific combination of components (i.e., the specific prostaglandin/retinoid combination) and the mode of administration. Generally, when orally administered, the prostaglandin is administered at a dose of 1 pg to 10 mg per day, desirably 10 pg to 1 mg per day, more desirably 1 to 500 μg per day, and most desirably 10 to 100 μg per day, while the retinoid is administered at a dose of 1 μg to 5 g per day, desirably 0.1 mg to 1 g per day, more desirably 1 to 100 mg per day, and most desirably 5 to 50 mg per day.

Generally, when administered by intravenous, intramuscular, or subcutaneous injection, the prostaglandin is administered at a dosage of 1 pg to 10 mg per day, desirably 10 pg to 1 mg per day, more desirably 1 to 500 μg per day, and most desirably 10 to 100 μg per day, while the retinoid is administered at a dosage of 1 μg to 5 g per day, desirably 0.1 mg to 1 g per day, more desirably 1 to 100 mg per day, and most desirably 5 to 50 mg per day.

Generally, when delivered by topical, transdermal, or ophthalmic application, or inhalation, rectal, or vaginal administration, the prostaglandin is administered at a dose of 1 pg to 100 mg per day, desirably 10 pg to 10 mg per day, more desirably 100 pg to 1 mg per day, and most desirably 0.01 to 0.5 mg per day, while the retinoid is administered at a dose of 50 ng to 500 mg per day, desirably 500 ng to 50 mg per day, more desirably 5 μg to 5 mg per day, and most desirably 50 to 500 μg per day.

The invention also features a method for identifying compounds useful for treating a patient having an immunoinflammatory disorder or a proliferative skin disease. The method includes the steps of: contacting immune cells in vitro with (i) a prostaglandin or a retinoid; and (ii) a candidate compound, and determining whether the immune response is modulated relative to (a) immune cells contacted with the prostaglandin or retinoid but not contacted with the candidate compound, and (b) immune cells contacted with the candidate compound but not with the prostaglandin or retinoid. A candidate compound that, when combined with the prostaglandin or retinoid, modulates the immune response to a greater degree than controls, is a compound that is potentially useful for treating a patient having an immunoinflammatory disorder or a proliferative skin disease.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

The term “immunoinflammatory disorder” encompasses a variety of conditions, including autoimmune diseases. Immunoinflammatory disorders result in the destruction of healthy tissue by an inflammatory process. Examples of immunoinflammatory disorders include rheumatoid arthritis, ulcerative colitis, Crohn's disease, stroke-induced brain cell death, septic shock syndrome, ankylosing spondylitis, fibromyalgia, asthma, multiple sclerosis, type I diabetes, systemic lupus erythematosus, scleroderma, systemic sclerosis, inflammatory dermatoses, myasthenia gravis, and Sjögren's syndrome. Inflammatory dermatoses include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

The term “proliferative skin disease” encompasses benign and malignant proliferative skin diseases that are characterized by accelerated cell division in the epidermis or dermis. Examples of proliferative skin diseases include psoriasis, atopic dermatitis, non-specific dermatitis, primary irritant contact dermatitis, allergic contact dermatitis, basal and squamous cell carcinomas of the skin, lamellar ichthyosis, epidermolytic hyperleratosis, premalignant keratosis, acne, and seborrheic dermatitis.

By “prostaglandin” is meant alprostidil, dinoprostone, misoprostil, prostaglandin E2, prostaglandin A1, prostaglandin A2, prostaglandin B1, prostaglandin B2, prostaglandin D2, prostaglandin Fla, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α, and prostaglandin J.

By “retinoid” is meant retinoic acid, retinol, and retinal, and natural or synthetic derivatives of retinoic acid, retinol, or retinal that are capable of binding to a retinoid receptor and consist of four isoprenoid units joined in a head-to-tail manner. Examples of retinoids include tretinoin, vitamin A2 (3,4-didehydroretinol), α-vitamin A (4,5-didehydro-5,6-dihydroretinol), 13-cis-retinol, 13-cis retinoic acid (isotretinoin), 9-cis retinoic acid (9-cis-tretinoin), 4-hydroxy all-trans retinoic acid, torularodin, methyl retinoate, retinaldehyde, 13-cis-retinal, etretinate, tazoretene, acetretin, alitretinoin and adapelene.

The combination of a prostaglandin and a retinoid for the treatment of immunoinflammatory disorders and proliferative skin diseases allows for the administration of a low dose of each compound and less total active compound, thus providing similar efficacy with less toxicity, and reduced costs.

Other features and advantages of the invention will be apparent from the following detailed description, and from the claims.

### DETAILED DESCRIPTION

We have discovered that the combination of the prostaglandin, alprostadil, with a retinoid, tretinoin, had substantial TNFα suppressing activity on white blood cells. Concentrations that effectively suppressed TNFα activity were not unacceptably toxic to normal cells. Thus, combinations of prostaglandins and retinoids are useful for the treatment of immunoinflammatory disorders and proliferative skin diseases.

Therapy

Combination therapy according to the invention may be performed alone or in conjunction with another therapy and may be provided at home, the doctor's office, a clinic, a hospital's outpatient department, or a hospital. Treatment generally begins at a hospital so that the doctor can observe the therapy's effects closely and make any adjustments that are needed. The duration of the combination therapy depends on the type of disease or disorder being treated, the age and condition of the patient, the stage and type of the patient's disease, and how the patient responds to the treatment. Additionally, a person having a greater risk of developing an immunoinflammatory disorder or proliferative skin disease (e.g., a person who is genetically predisposed or having a prior diagnosis of an immunoinflammatory or proliferative skin disorder) may receive prophylactic treatment to inhibit or delay the onset of symptoms.

A proliferative skin disease is alleviated when there is a noticeable decrease in the size or thickness of a lesion to palpation. This decrease can occur either with or without residual redness, dilated blood vessels, hyper-pigmentation, or hypo-pigmentation. For the purposes of this invention, psoriasis is considered alleviated when a scale-free psoriasis lesion is noticeably decreased in thickness.

The dosage, frequency and mode of administration of each component of the combination can be controlled independently. For example, one compound may be administered orally three times per day, while the second compound may be administered intramuscularly once per day. Combination therapy may be given in on-and-off cycles that include rest periods so that the patient's body has a chance to recovery from any as yet unforeseen side-effects. The compounds may also be formulated together such that one administration delivers both compounds.

Formulation of Pharmaceutical Compositions

Suitable modes of administration include oral, rectal, intravenous, intramuscular, subcutaneous, inhalation, topical or transdermal, vaginal, and ophthalmic. Administration of each compound of the combination may be by any suitable means that results in a concentration of the compound that, combined with the other compound, is effective. Each compound can be admixed with a suitable carrier substance, and is generally present in an amount of 1-95% by weight of the total weight of the composition. The pharmaceutical compositions may be formulated according to conventional pharmaceutical practice (see, e.g., Remington: The Science and Practice of Pharmacy, (20th ed.) ed. A. R. Gennaro, 2000, Lippencott Williams & Wilkens, Philadelphia, Pa., and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release formulations.

Administration of compounds in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Topical Compositions

Therapeutic compositions suitable for topical application include conventional anhydrous or aqueous preparations including ointments, lotions, creams, pastes, jellies, sprays, aerosols, and oils. There preparations can include oleaginous, aqueous, or emulsion-type bases. Optionally, topically applied formulations can be covered with an occlusive or semi-occlusive dressing.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Dosages

The dosage of each compound of the claimed combinations used in any given therapeutic method depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

As is described above, the compound(s) may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. Below, the dosages for prostaglandins and retinoids are described.

For oral, intramuscular, subcutaneous, and intravenous administration of the prostaglandin, the dosage is normally about 1 pg to 10 mg per day, desirably about 10 pg to 1 mg per day, more desirably about 1 to 500 μg per day, and most desirably about 10 to 100 μg per day. Administration can be one to four times daily for one day to one year, and may even be for the life of the patient. Chronic, long-term administration will be indicated in many cases. For topical, inhalation, rectal, vaginal and ophthalmic administration of the prostaglandin, the dosage is normally about 1 pg to 100 mg per day, desirably about 1 pg to 10 mg per day, more desirably about 100 pg to 1 mg per day, and most desirably about 0.01 to 0.5 mg per day. Administration can be one to four times daily. Systemic dosing will result in steady-state plasma concentrations of about 1 picomolar to 1 micromolar, more desirably about 100 picomolar to 100 nanomolar, and most desirably about 1 to 10 nanomolar.

For oral, intramuscular, subcutaneous, and intravenous administration of the retinoid, the dosage is about 1 μg to 5 g per day, desirably about 0.1 mg to 1 g mg per day, more desirably about 1 to 100 mg per day, and most desirably about 5 to 50 mg per day. For topical, inhalation, rectal, vaginal, or ophthalmic administration, the dosage is about 50 ng to 500 mg per day, desirably 500 ng to 50 mg per day, more desirably about 5 μg to 5 mg per day, and most desirably 50 to 500 μg per day. Administration can be one to four times daily for one day to one year, and may even be for the life of the patient. Chronic, long-term administration will be indicated in many cases. Systemic dosing of tretinoin, for example, results in steady-state plasma concentration desirably of 500 picomolar to 50 micromolar, more desirably 5 nanomolar to 5 micromolar, and most desirably 50 to 500 nanomolar.

The following examples are to illustrate the invention. They are not meant to limit the invention in any way.

### EXAMPLE 1

### Preparation of Combinations of Compounds

Stock solutions at 1.6 mg/ml of alprostadil, and 4.0 mg/ml of tretinoin acetate (Sigma-Aldrich, St. Louis, Mo.; catalog numbers P5515 and R-2625, respectively) were made in dimethylsulfoxide (DMSO). The alprostadil master plates were made by adding 25 μl of the concentrated stock solution to columns 3, 9, and 15 (rows C through N) of a polypropylene 384-well storage plate that had been pre-filled with 75 μl of anhydrous DMSO. Using a TomTec Quadra Plus liquid handler, the 25 μl of alprostadil stock solution was serially diluted four times into the adjacent columns (columns 4-7, 10-13, 16-19). The sixth column (8, 14, and 20) did not receive any compound and served as a vehicle control. The tretinoin master plates were made by adding 25 μl of the concentrated stock solution to the appropriate wells (row C, columns 3-8; row C, columns 9-14; row C, columns 15-20; row I, columns 3-8; row I, columns 9-14; row I, columns 15-20) of the appropriate master polypropylene 384-well storage plate. These master plates had been pre-filled with 75 μl of anhydrous DMSO. Using the TomTec Quadra Plus liquid handler, the 25 μl was serially diluted four times in the adjacent rows (rows D-G, and J-M). The sixth row (H and N) did not receive any compound to serve as a vehicle control. Master plates were sealed and stored at −20° C. until ready for use.

The final alprostadil/tretinoin combination plates were generated by transferring 1 μl from each of the alprostadil and tretinoin master plates to a dilution plate containing 100 μl of media (RPMI; Gibco BRL, #11875-085), 10% Fetal Bovine Serum (Gibco BRL, #25140-097), 2% Penicillin/Streptomycin (Gibco BRL, #15140-122)) using the TomTec Quadra Plus liquid handler. This dilution plate was then mixed and a 10 μl aliquot transferred to the final assay plate, which had been pre-filled with 40 μl/well RPM media containing the appropriate stimulant to activate TNFα secretion (see below).

### EXAMPLE 2

### Assay for TNFα Suppressing Activity by the Combination of Alprostadil and Tretinoin

The compound dilution matrix was assayed using a TNFα ELISA method. Briefly, a 100 μl suspension of diluted human white blood cells contained within each well of a polystyrene 384-well plate (NalgeNunc) was stimulated to secrete TNFα by treatment with a final concentration of 10 ng/ml phorbol 12-myristate 13-acetate (Sigma) and 750 ng/ml ionomycin (Sigma). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-TNFα antibody (PharMingen, #18631D). After a two-hour incubation, the plate was washed (Tecan PowerWasher 384) with phosphate buffered saline (PBS) containing 0.1% Tween 20 (polyoxyethylene sorbitan monolaurate) and incubated for an additional one hour with another anti-TNFα antibody that was biotin labeled (PharMingen, 18642D) and horseradish peroxidase (HRP) coupled to strepavidin (PharMingen, #13047E).). After the plate was washed with 0.1% Tween 20/PBS, an HRP-luminescent substrate was added to each well and light intensity measured using a LJL Analyst plate luminometer. Sets of control wells contained a serial dilution of Cyclosporin A (Sigma) starting at a final concentration of 0.5 μg/ml.

Low doses of alprostadil significantly increased the ability of tretinoin to suppress TNFα secretion from stimulated white blood cells. As seen in Table 1, tretinoin alone maximally inhibited TNFα secretion by 36% at concentrations ranging from 0.83-13.3 μM. The combination of 0.052 μM tretinoin and 0.004 μM alprostadil was able to suppress TNFα secretion by 42%. The shift from 0.83 to 0.052 μM tretinoin without any loss of activity represents a 16-fold increase in tretinoin potency in the presence of low-dose alprostadil. Maximal tretinoin efficacy (−36% TNFα suppression) is therefore maintained while reducing the concentration of total drug species by greater than 90% (0.052 μM tretionoin+0.004 μM alprostadil compared to 0.832 μM tretinoin alone). Table 1 also demonstrates that low-dose tretinoin potentiated TNFα suppression by alprostadil. 1.130 M alprostadil inhibited TNFα secretion by 57%. The same level of inhibition was achieved by 0.071 μM alprostadil in the presence of 0.052 μM tretinoin. The 0.123 uM concentration of the low-dose alprostadil/low-dose tretinoin combination represents an 89% reduction in total drug species when compared to 1.130 μM alprostadil alone.

Data from a secondary screen (Table 2) confirm and extend the observed synergism between alprostadil and tretinoin. In this experiment, white blood cells were stimulated using 1 μg/ml LPS. TNFα inhibition by tretinoin alone was approximately 50%, at concentrations from 6.7-13.3 μM. 59% inhibition of TNFα secretion was achieved by only 0.052 μM tretinoin in the presence of 0.004 μM alprostadil. The data also show that the TNFα-suppressing activity of 0.208 μM tretinoin (32.5%) was doubled (64.0%) by the addition of 0.004 μM alprostadil. This level of inhibition was not attainable by less than 0.035 μM alprostadil alone. Further evidence for the enhancement of alprostadil effects by tretinoin were also measured. The maximal TNFα-inhibitory effect (82%) observed for alprostadil alone required a concentration of at least 1.128 μM. This level of inhibition was exceeded (86%) by only 0.282 μM alprostadil in the presence of 0.104 μM tretinoin.

TABLE 1

| | Alprostadil [μM] | | | | | |
|---|---|---|---|---|---|---|
| Tretinoin [μM] | 1.130 | 0.283 | 0.071 | 0.018 | 0.004 | 0.000 |
| 13.316 | 80.62 | 79.57 | 72.73 | 73.92 | 62.87 | 36.21 |
| 3.329 | 72.04 | 69.23 | 64.19 | 62.15 | 52.87 | 34.12 |
| 0.832 | 70.58 | 68.71 | 65.20 | 57.69 | 54.71 | 34.53 |
| 0.208 | 68.35 | 65.45 | 59.56 | 54.48 | 48.85 | 28.70 |
| 0.052 | 63.46 | 64.65 | 56.79 | 50.39 | 41.65 | 13.83 |
| 0.000 | 56.80 | 53.80 | 50.52 | 39.75 | 33.30 | 0.00 |

TABLE 2
Alprostadil [μM]

| Tretinoin [μM] | 1.128 | 0.564 | 0.282 | 0.141 | 0.071 | 0.035 | 0.018 | 0.009 | 0.004 | 0.000 |
|---|---|---|---|---|---|---|---|---|---|---|
| 13.316 | 94.86 | 94.58 | 91.44 | 89.43 | 85.35 | 84.32 | 79.81 | 76.18 | 72.63 | 52.08 |
| 6.658 | 93.50 | 91.23 | 88.53 | 88.56 | 84.81 | 78.32 | 68.70 | 72.21 | 68.94 | 48.68 |
| 3.329 | 90.33 | 88.60 | 88.27 | 83.21 | 68.40 | 77.24 | 71.06 | 69.60 | 63.24 | 44.10 |
| 1.664 | 89.37 | 89.34 | 78.92 | 84.76 | 77.01 | 75.18 | 73.03 | 67.84 | 67.16 | 37.58 |
| 0.832 | 36.92 | 90.73 | 86.76 | 79.58 | 83.87 | 77.24 | 74.59 | 69.21 | 65.26 | 38.38 |
| 0.416 | 91.20 | 89.29 | 86.39 | 77.04 | 73.74 | 76.37 | 67.45 | 71.11 | 63.82 | 32.69 |
| 0.208 | 87.47 | 90.44 | 88.16 | 83.41 | 70.91 | 73.53 | 62.12 | 69.30 | 64.01 | 32.50 |
| 0.104 | 85.01 | 75.99 | 86.00 | 75.94 | 78.26 | 67.56 | 70.92 | 61.99 | 61.37 | 35.08 |
| 0.052 | 89.45 | 79.22 | 78.62 | 73.82 | 75.04 | 69.51 | 68.82 | 65.83 | 58.84 | 32.71 |
| 0.000 | 81.60 | 79.45 | 75.66 | 74.28 | 71.50 | 57.32 | 54.78 | 49.17 | 44.61 | 0.33 |

### Other Embodiments

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cellular and molecular biology, pharmacology, endocrinology, or related fields are intended to be within the scope of the invention.

1. A method for treating a patient who has an immunoinflammatory disorder or a proliferative skin disease, or is at risk for developing an immunoinflammatory disorder or a proliferative skin disease, said method comprising administering to said patient a prostaglandin and a retinoid, wherein the prostglandin and the retinoid are administered simultaneously or within 10 days of each other, in amounts sufficient to treat said patient.

2. The method of claim 1, wherein said prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α or prostaglandin J.

3. The method of claim 2, wherein said prostaglandin is alprostadil or misoprostil.

4. The method of claim 1, wherein said retinoid is tretinoin, retinal, retinol, vitamin A2, α-vitamin A, 13-cis-retinol, isotretinoin, 9-cis-tretinoin, 4-hydroxy all-trans retinoic acid, torularodin, methyl retinoate, retinaldehyde, 13-cis-retinal, etretinate, tazoretene, acetretin, alitretinoin or adapelene.

5. The method of claim 4, wherein said retinoid is tretinoin.

6. The method of claim 1, wherein said prostaglandin is alprostidil and said retinoid is tretinoin.

7. The method of claim 1, wherein said prostaglandin and said retinoid are administered within 5 days of each other.

8. The method of claim 7, wherein said prostaglandin and said retinoid are administered within 24 hours of each other.

9. The method of claim 8, wherein said prostaglandin and said retinoid are administered within one hour of each other.

10. The method of claim 9, wherein said prostaglandin and said retinoid are administered simultaneously.

11. The method of claim 1, wherein said immunoinflammatory disorder is rheumatoid arthritis, psoriasis, ulcerative colitis, Crohn's disease, an inflammatory dermatosis, septic shock syndrome, or stroke induced brain cell death.

12. The method of claim 1, wherein said inflammatory dermatosis is psoriasis.

13. The method of claim 1, wherein said proliferative skin disease is contact dermatitis or acne.

14. The method of claim 1, wherein said prostaglandin and said retinoid are administered to said patient by intravenous, intramuscular, subcutaneous, rectal, oral, topical, intravaginal, ophthalmic or inhalation administration.

15. The method of claim 14, wherein said prostaglandin is administered in an amount of 1 pg to 100 mg per day, and said retinoid is administered in an amount of 50 ng to 5 g per day.

16. The method of claim 15, wherein said prostaglandin is administered in an amount of 10 pg to 10 mg per day, and said retinoid is administered in an amount of 500 ng to 1 g per day.

17. The method of claim 16, wherein said prostaglandin is administered in an amount of 100 pg to 1 mg per day, and said retinoid is administered in an amount of 5 μg to 100 mg per day.

18. The method of claim 17, wherein said prostaglandin is administered in an amount of 0.01 ng to 0.5 mg per day, and said retinoid is administered in an amount of 50 μg to 50 mg per day.

19. A pharmaceutical composition comprising a prostaglandin, a retinoid, and a pharmaceutically acceptable carrier, wherein said prostaglandin and said retinoid are each present in amounts that, when administered together to a patient having an immunoinflammatory disorder, inhibit or reduce immunoinflammation or dermal/epidermal proliferation.

20. The pharmaceutical composition of claim 19, wherein said prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α, or prostaglandin J.

21. The pharmaceutical composition of claim 20, wherein said prostaglandin is alprostidil or misoprostil.

22. The pharmaceutical composition of claim 19, wherein said retinoid is tretinoin, retinal, retinol, vitamin A2, α-vitamin A, 13-cis-retinol, isotretinoin, 9-cis-tretinoin, 4-hydroxy all-trans retinoic acid, torularodin, methyl retinoate, retinaldehyde, 13-cis-retinal, etretinate, tazoretene, acetretin, alitretinoin or adapelene.

23. The pharmaceutical composition of claim 22, wherein said retinoid is tretinoin or retinol.

24. The pharmaceutical composition of claim 19, wherein said prostaglandin is alprostidil and said retinoid is tretinoin or retinol.

25. The pharmaceutical composition of claim 19, wherein said prostaglandin and said retinoid are suitable for intravenous, intramuscular, subcutaneous, rectal, oral, topical, intravaginal, ophthalmic or inhalation administration.

26. A pharmaceutical pack comprising a prostaglandin and a retinoid.

27. The pharmaceutical pack of claim 26, wherein said prostaglandin is alprostidil, misoprostil, dinoprostone, prostaglandin E2, prostaglandin A1, prostaglandin A2, prostaglandin B 1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α, or prostaglandin J.

28. The pharmaceutical pack of claim 26, wherein said retinoid is tretinoin, retinal, retinol, vitamin A2, α-vitamin A, 13-cis-retinol, isotretinoin, 9-cis-tretinoin, 4-hydroxy all-trans retinoic acid, torularodin, methyl retinoate, retinaldehyde, 13-cis-retinal, etretinate, tazoretene, acetretin, alitretinoin or adapelene.

29. The pharmaceutical pack of claim 26, wherein said prostaglandin and said retinoid are formulated separately and in individual dosage amounts.

30. The pharmaceutical pack of claim 26, wherein said prostaglandin and said retinoid are formulated together and in individual dosage amounts.

31. A method for identifying combinations of compounds useful for treating a patient having an immunoinflammatory disorder or proliferative skin disease, said method comprising the steps of:
(a) contacting white blood cells in vitro with (i) a prostaglandin or a retinoid, and (ii); and a candidate compound; and
(b) determining whether the combination of said prostaglandin or retinoid and said candidate compound reduces TNFα levels in said white blood cells relative to white blood cells contacted with said prostaglandin or retinoid but not contacted with said candidate compound, or white blood cells contacted with said candidate compound but not with said prostaglandin or retinoid, wherein a reduction of said TNFα levels identifies said combination as a combination that is useful for treating a patient having an immunoinflammatory disorder or proliferative skin disease.
